(19) United States
(12) Patent Application Publication
(10) Pub. No.: US 2006/0178711 A1
(43) Pub. Date: Aug. 10, 2006

(54) Prosthetic hearing implant fitting technique

(75) Inventors: James F. Patrick, Roseville (AU); John L. Parker, Roseville (AU)

Correspondence Address: JAGTIANI + GUTTAG, 10363-A DEMOCRACY LANE, FAIRFAX, VA, 22030, US

(73) Assignee: Cochlear Limited, Lane Cove (AU)

(21) Appl. No.: 11/348,309

(22) Filed: Feb. 7, 2006

Related U.S. Application Data
(60) Provisional application No. 60/650,148, filed on Feb. 7, 2005.

(51) Int. Cl. A61N 1/18

(52) U.S. Cl. 607/57

(57) ABSTRACT

A fitting system and method for fitting a prosthetic hearing implant for a recipient, the implant having an array of electrodes implanted in the recipient's a cochlear. The fitting system comprises: a user interface configured to provide acoustic-based fitting data and receive acoustic-based control inputs; an acoustic test signal generator configured to provide the recipient acoustic test signals generated in response to said acoustic-based control inputs; and a data transformer configured to transform said acoustic-based fitting data to implant-based fitting data for use by the hearing implant.

### CROSS-REFERENCE TO RELATED APPLICATIONS

This application claims the benefit of U.S. Provisional Application No. 60/650,148, entitled “Hearing Implant Programming Technique,” filed Feb. 7, 2005, which is hereby incorporated by reference herein in its entirety.

### BACKGROUND

1. Field of the Invention

The present invention relates generally to prosthetic hearing implants, and more particularly, to fitting prosthetic hearing implants.

2. Related Art

There are many medical implants that deliver electrical stimulation to a patient or recipient (“recipient” herein) for a variety of therapeutic benefits. For example, prosthetic hearing implants have been developed to provide persons with sensorineural hearing loss with the ability to perceive sound. The hair cells of the cochlea of a normal healthy ear converts acoustic signals into nerve impulses. People who are profoundly deaf due to the absence or destruction of cochlea hair cells are unable to derive suitable benefit from conventional hearing aid systems. Prosthetic hearing implants have been developed to provide such persons with the ability to perceive sound.

Prosthetic hearing implants typically comprise external and implanted or internal components that cooperate with each other to provide sound sensations to a recipient. The external component traditionally includes a microphone that detects sounds, such as speech and environmental sounds, a speech processor that selects and converts certain detected sounds, particularly speech, into a coded signal, a power source such as a battery, and an external transmitter antenna.

The coded signal output by the speech processor is transmitted transcutaneously to an implanted receiver/stimulator unit, commonly located within a recess of the temporal bone of the recipient. This transcutaneous transmission occurs via the external transmitter antenna which is positioned to communicate with an implanted receiver antenna disposed within the receiver/stimulator unit. This communication transmits the coded sound signal while also providing power to the implanted receiver/stimulator unit. Conventionally, this link has been in the form of a radio frequency (RF) link, although other communication and power links have been proposed and implemented with varying degrees of success.

The implanted receiver/stimulator unit also includes a stimulator that processes the coded signal and outputs an electrical stimulation signal to an intra-cochlea electrode assembly mounted to on carrier member. The electrode assembly typically has a plurality of electrodes that apply electrical stimulation to the auditory nerve to produce a hearing sensation corresponding to the original detected sound. Because the cochlea is tonotopically mapped, that is, partitioned into regions each responsive to stimulus signals in a particular frequency range, each electrode of the implantable electrode array delivers a stimulating signal to a particular region of the cochlea. In the conversion of sound to electrical stimulation, frequencies are allocated to individual electrodes of the electrode assembly that lie in positions in the cochlea that are close to the region that would naturally be stimulated in normal hearing. This enables the prosthetic hearing implant to bypass the hair cells in the cochlea to directly deliver electrical stimulation to auditory nerve fibers, thereby allowing the brain to perceive hearing sensations resembling natural hearing sensations.

The effectiveness of a prosthetic hearing implant is dependent, not only on the device itself, but also on the way in which the device is fit. Fitting of a device, also referred to as “programming” or “mapping,” creates a set of instructions that defines the specific characteristics used to stimulate the electrodes of the implanted array. This set of instructions is referred to as the recipient's “program” or “map.”

Advances in cochlear implant technology have resulted in a relatively complex fitting process. Today's cochlear implants offer a number of sophisticated parameters that can be manipulated to improve sound quality and speech understanding. As such, implant programming is performed by an audiologist with specialized training in the field of cochlear implants. Typically, the audiologist uses interactive software and computer hardware to create individualized program that are downloaded to the recipient's speech processor for real-time use.

### SUMMARY

Aspects of the present invention are generally directed to a technique for fitting a prosthetic hearing implant which can be performed by an audiologist using conventional acoustic-based data displays, control inputs and acoustic test signals to determine the electrical stimulation parameters required to operate the hearing implant.

In one aspect of the invention, a fitting system for fitting a prosthetic hearing implant for a recipient, the implant having an array of electrodes implanted in the recipient's a cochlear. The fitting system comprises: a user interface configured to provide acoustic-based fitting data and receive acoustic-based control inputs; an acoustic test signal generator configured to provide the recipient acoustic test signals generated in response to said acoustic-based control inputs; and a data transformer configured to transform said acoustic-based fitting data to implant-based fitting data for use by the hearing implant.

### BRIEF DESCRIPTION OF THE DRAWINGS

Embodiments of the present invention are described herein in conjunction with the accompanying drawings, in which:

FIG. 1 is an exemplary hearing prosthesis which may be advantageously implemented with embodiments of the present invention;

FIG. 2 is a schematic diagram illustrating one exemplary cochlear implant fitting arrangement in which one embodiment of a hearing implant fitting system configured in accordance with the teachings of the present invention is implemented to fit a cochlear implant to a recipient;

FIG. 3 is a high-level flow chart illustrating operations that may be performed while fitting the cochlear implant illustrated in FIG. 1 utilizing the fitting system illustrated in FIG. 2;

FIG. 4 is a high-level functional block diagram of one embodiment of the hearing implant fitting system illustrated in FIG. 2;

FIG. 5A is a graph illustrating a pure tone acoustic test signal;

FIG. 5B is a graph illustrating a composition acoustic test signal comprising a broad range of frequencies;

FIG. 6 represents an exemplary frequency-gain data display displayed by a display device in one exemplary embodiment of the present invention to provide an audiologist with threshold and comfort levels in an acoustic-based display format;

FIG. 7A is a line graph illustrating the relationship between frequency and electrode according to one embodiment of the present invention; and

FIG. 7B is a line graph illustrating the relationship between amplitude and stimulus current according to one embodiment of the present invention.

### DETAILED DESCRIPTION

Aspects of the present invention are generally directed to a technique for fitting a prosthetic hearing implant which can be performed by an audiologist using conventional acoustic-based data displays, control inputs and acoustic test signals to determine the electrical stimulation parameters required to operate the hearing implant. Embodiments of the present invention may relieve the audiologist of the need to explicitly set and map the electrical stimulation levels. Such setting and mapping of electrical stimulation involves procedures that may be unfamiliar to practitioners who are largely trained to fit conventional hearing aids using acoustic test signals. Hence, embodiments of the present invention assist those practitioners to perform the task of fitting a prosthetic hearing implant.

Prosthetic hearing implants include but are not limited to hearing aids, auditory brain stimulators, and Cochlear™ implants (also commonly referred to as Cochlear™ prostheses, Cochlear™ devices, Cochlear™ implant devices, and the like; generally and collectively referred to as “cochlear implants” herein). Embodiments of the present invention are described herein primarily in connection with one type of prosthetic hearing implant, a cochlear implant.

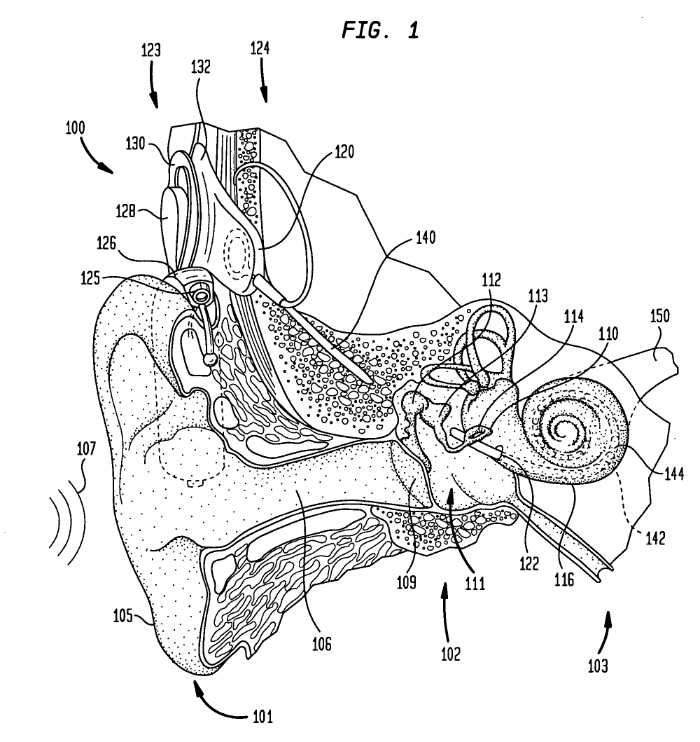

Cochlear implants use direct electrical stimulation of auditory nerve cells to bypass absent or defective hair cells that normally transduce acoustic vibrations into neural activity. Such devices generally use an electrode array inserted into the scala tympani of the cochlea so that the electrodes may differentially activate auditory neurons that normally encode differential pitches of sound. Auditory brain stimulators are used to treat a smaller number of recipients with bilateral degeneration of the auditory nerve. For such recipients, the auditory brain stimulator provides stimulation of the cochlear nucleus in the brainstem, typically with a planar electrode array; that is, an electrode array in which the electrode contacts are disposed on a two dimensional surface that can be positioned proximal to the brainstem. FIG. 1 is a perspective view of an exemplary cochlear implant 100 in which the programming techniques of the present invention may be employed.

FIG. 1 is a cut-away view of the relevant components of outer ear 101, middle ear 102 and inner ear 103, which are described next below. In a fully functional ear, outer ear 101 comprises an auricle 105 and an ear canal 106. An acoustic pressure or sound wave 107 is collected by auricle 105 and channeled into and through ear canal 106. Disposed across the distal end of ear cannel 106 is a tympanic membrane 109 which vibrates in response to acoustic wave 107. This vibration is coupled to oval window or fenestra ovalis 110 through three bones of middle ear 102, collectively referred to as the ossicies 111 and comprising the malleus 112, the incus 113 and the stapes 114. Bones 112, 113 and 114 of middle ear 102 serve to filter and amplify acoustic wave 107, causing oval window 110 to articulate, or vibrate. Such vibration sets up waves of fluid motion within cochlea 116. Such fluid motion, in turn, activates tiny hair cells (not shown) that line the inside of cochlea 116. Activation of the hair cells causes appropriate nerve impulses to be transferred through the spiral ganglion cells and auditory nerve 150 to the brain (not shown), where they are perceived as sound. In deaf persons, there is an absence or destruction of the hair cells. Prosthetic hearing implant 120 is needed to directly stimulate the ganglion cells to provide a hearing sensation to the recipient.

FIG. 1 also shows how an implanted prosthetic hearing implant 120 is positioned in relation to outer ear 101, middle ear 102 and inner ear 103. Prosthetic hearing implant 120 comprises external component assembly 123 which is directly or indirectly attached to the body of the recipient, and an internal component assembly 124 which is temporarily or permanently implanted in the recipient. External assembly 123 comprises microphone 125 for detecting sound which is outputted to a BTE (Behind-The-Ear) speech processing unit 126 that generates coded signals and are provided to an external transmitter unit 128, along with power from a power source such as a battery (not shown). External transmitter unit 128 comprises an external coil 130 and, preferably, a magnet (also not shown) secured directly or indirectly in external coil 130. Internal components 124 comprise an internal receiver unit 132 having an internal coil (not shown) that receives and transmits power and coded signals from external assembly 123 to a stimulator unit 120 to apply the coded signal along an electrode assembly 140. Electrode assembly 140 enters cochlea 116 at cochleostomy region 112 and has one or more electrodes 142 is positioned to substantially be aligned with portions of cochlea 116.

Cochlea 116 is tonotopically mapped with each region of the cochlea being responsive to acoustic and/or stimulus signals in a particular frequency range. To accommodate this property of cochlea 116, prosthetic hearing implant 100 include an array of electrodes each constructed and arranged to deliver appropriate stimulating signals to particular regions of the cochlea, each representing a different frequency component of a received audio signal. Signals generated by stimulator unit 120 are applied by the electrodes 142 of electrode array 144 to cochlea 116, thereby stimulating the auditory nerve 116. It should be appreciated that although in the embodiment shown in FIG. 1 electrodes 142 are arranged in an array 144, other arrangements are possible.

In one example, electrode array 144 may include a plurality of independent electrodes 142 each of which may be independently stimulated. For example, in an embodiment, employing Cochlear's Nucleus 24 system, electrode array 144 includes 22 independent electrodes each of which stimulates an area of the auditory nerve 150 of the recipient's cochlea 116. As one of ordinary skill in the art is aware, low-frequency sounds stimulate the basilar membrane most significantly at its apex, while higher frequencies more strongly stimulate the basilar membrane's base. Thus, electrodes 142 of electrode array 144 located near the base of the cochlea are used to simulate high frequency sounds while electrodes closer to the apex are used to simulate lower frequency sounds. Typically, in such a system, speech processing unit 126 stimulates only the electrodes with the largest signals. For example, cochlear implant 100 may estimate the outputs for each of the 22 electrodes 142 and select the ones with the largest amplitude (that is, maxima). The number of maxima selected may vary, for example, between five (5) and ten (10), depending on a variety of factors. Moreover, the rate of stimulation, often referred to in units of pulses per second, may also vary. Each of the applied maxima will be referred to herein as a channel of stimulation (or stimulation channel). Thus, in an example in which eight (8) maxima are applied, the system will be described as applying eight (8) channels of stimulation.

As one of ordinary skill in the art will appreciate, the present invention may be used in combination with any speech strategy now or later developed including, but not limited to, Continuous Interleaved Sampling (CIS), Spectral PEAK Extraction (SPEAK), and Advanced Combination Encoders (ACE™). An example of such speech strategies is described in U.S. Pat. No. 5,271,397, the entire contents and disclosures of which is hereby incorporated by reference herein. Other examples also may also include front-end processing algorithms such as those described in U.S. Pat. No. 6,731,767 entitled ‘Adaptive dynamic range of optimization sound processor,’ WO 2005/006808 entitled ‘Method and Device for Noise Reduction’. Moreover, a genetic algorithm may be used to optimize the map for features such as, but not limited to: rate, growth function and the like, as described in WO 2004/080532 entitled ‘Cochlear implant System with Map Optimization Using a Genetic Algorithm. The above references are hereby incorporated by reference herein in their entireties. The present invention may also be used with other speech coding strategies now or later developed. Certain embodiments of the present invention may be used on Cochlear Limited's Nucleus™ implant system that uses a range of coding strategies alternatives, including SPEAK, ACE™, and CIS.

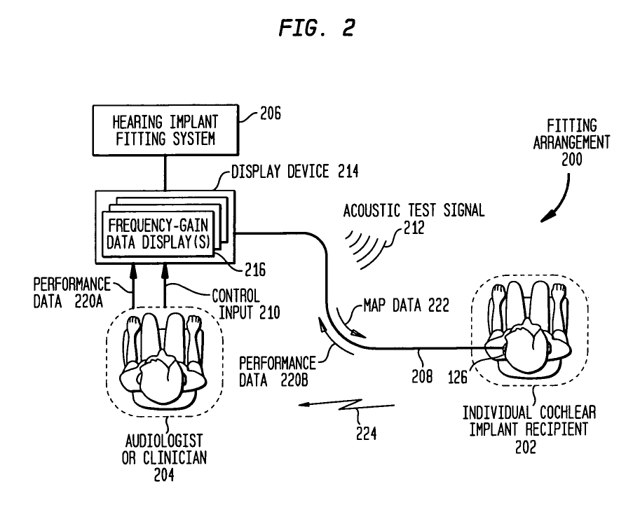

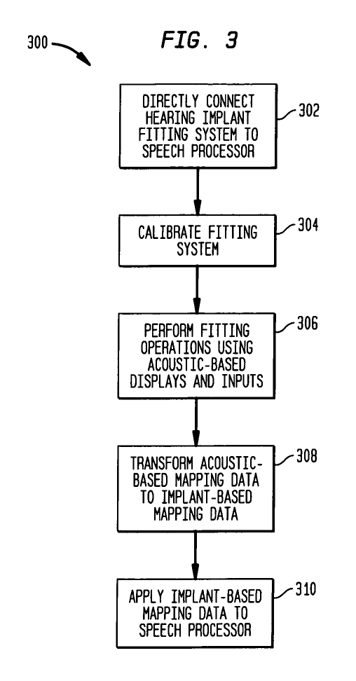

FIG. 2 is a schematic diagram illustrating one exemplary arrangement 200 in which a hearing implant fitting system 206 configured in accordance with the teachings of the present invention may be implemented. FIG. 3 is a high-level flow chart illustrating operations that may be performed while fitting cochlear implant 100 utilizing the fitting system illustrated in FIG. 2.

As one of ordinary skill in the art would appreciate, the characteristics and code transmitted by cochlear implant 100 are dependent in part on the effectiveness with which the implant is fit to an individual recipient 202. Fitting of cochlear implant 100 (also commonly referred to as “programming” or “mapping”) creates a set of instructions (data or code; “mapping data” 222 herein) that defines the specific characteristics used to stimulate electrodes 142 of the implanted electrode array. This set of instructions is commonly referred to as the recipient's “program” or “map.”

As shown in FIG. 2, an audiologist or clinician 204 uses a hearing implant fitting system 206 (“fitting system” herein) comprising interactive software and computer hardware to create individualized recipient map data 222 that are digitally stored on system 206 and ultimately downloaded to the memory of speech processor 126 of recipient 202. System 206 is programmed and/or implements software programmed to carry out one or more of the functions of mapping, neural response measuring, acoustic stimulating, and recording of neural response measurements and other stimuli.

In the embodiment illustrated in FIG. 2, speech processor 126 of cochlear implant 100 is connected directly to fitting system 206 to establish a data communication link 208 between the speech processor and fitting system. This is one of the initial operations performed in a fitting process, as shown in block 302 of FIG. 3. System 206 is thereafter bi-directionally coupled by means of data communication link 208 with speech processor 126. It should be appreciated that although speech processor 126 and fitting system 206 are connected via a cable in FIG. 2, any communications link now or later developed may be utilized to communicably couple the implant and fitting system.

A calibration of electrodes 142 is performed at block 304, including testing the impedance level of each such electrode. This calibration may include the presentation of performance data 220A or 220B as required by the implemented fitting protocol.

Once cochlear implant 100 is calibrated, specific mapping data 222 is determined, as shown in block 306 of FIG. 3B. The particular details of the implemented fitting process are specific to the recipient, cochlear implant manufacturer, cochlear implant device, etc. As a result, only selected exemplary mapping data are described herein for clarity.

Today, most cochlear implants require at least two values to be set for each stimulating electrode 142. These values are referred to as the Threshold level (commonly referred to as the “THR” or “T-level;” “threshold level” herein) and the Maximum Comfortable Loudness level (commonly referred to as the Most Comfortable Loudness level, “MCL,” “M-level,” or “C;” simply “comfort level” herein). Threshold levels are comparable to acoustic threshold levels; comfort levels indicate the level at which a sound is loud but comfortable. It should be appreciated that although the terminology and abbreviations are device-specific, the general purpose of threshold and comfort levels is common across all cochlear implants: to determine a recipient's electrical dynamic range.

Because of the currently common usage of threshold and current levels, exemplary embodiments of the present invention are described herein in the context of determining such values for cochlear implant 100. As one of ordinary skill in the art would appreciate, however, the present invention may be used to perform any fitting operations for any prosthetic hearing implant now or later developed.

Advances in cochlear implant technology have resulted in a relatively complex fitting process. Today's cochlear implants offer a number of sophisticated parameters that can be manipulated to improve sound quality and speech understanding. As noted, embodiments of the present invention are generally directed to fitting a prosthetic hearing implant such as cochlear implant 100 which can be performed by an audiologist using conventional acoustic input test signals. In contrast to conventional approaches in which implant programming is performed by an audiologist with specialized training in the field of cochlear implants, the present invention enables cochlear implants to be implanted by an audiologist or clinician lacking such specialized knowledge. As will be described in detail below, this is achieved by providing the audiologist or clinician with a user interface that provides and receives acoustic-based data; that is, data which is in the form commonly used by audiologists to fit hearing aids. Typically, such data is presented as one or more graphs or plots 216 illustrating frequency-gain relationships, although other presentations are feasible. In FIG. 2, such frequency-gain data display(s) 216 are shown presented on a display device 214, although any apparatus for presenting such data may be utilized. As such, fitting system 200 is referred to herein as having a user interface that presents data in the “acoustic domain.” By enabling the audiologist or clinician to operate fitting system 200 in the acoustic domain, they are thereby relieved of the need to explicitly and directly set electrical stimulation levels for a particular cochlear implant or other prosthetic hearing implant. This is described in further detail below.

Threshold levels may be obtained using an ascending presentation, followed by a standard bracketing procedure. Comfort levels are commonly obtained through a method referred to as loudness scaling. In contrast to conventional approaches in which the level of current is gradually increased, in the present invention, the level of the acoustic test signal 212 is increased. This occurs while recipient 202 reports on the level of loudness and comfort.

In adult cochlear implant patients, threshold and comfort levels are typically measured using verbal feedback from recipient 202. For children, who often lack the listening experience, language, or conceptual development to perform specific fitting tasks, audiologists and clinicians must often rely on clinical intuition and trial and error to appropriately estimate comfort levels for young recipients. The above and other feedback is generally referred to by reference numeral 224 in FIG. 2. Performance data provided directly to fitting system 206 may be provided via data connection 208 as performance data 220B, while performance data provided by the audiologist/clinician based on oral feedback or observations 224 is shown in FIG. 2 as performance data 220A (performance data 220A and 220B is generally and collectively referred to herein as performance data 220).

At block 308 of FIG. 3, fitting system 206 transforms the fitting data from the acoustic domain to the domain of the particular cochlear implant 100. In one embodiment described in detail below, threshold and comfort levels are psychophysical judgments of loudness that are measured in clinical units of electrical current, referred to as current units (cu). In the exemplary embodiment described herein, there is a single frequency channel allocated to each electrode 142 of electrode array 144. As such, mapping data 222 includes threshold and comfort levels (in cu's) for each electrode 142 which, as noted, is allocated to a particular frequency band. This transformation process is described in further detail below. The mapping data 222 is downloaded from fitting system 206 to cochlear implant 100 at block 310. As a result, from the audiologist's point of view, this fitting process is similar to the fitting of conventional hearing aids, wherein an audiologist measures hearing threshold and comfort levels across the audible frequency range (125-8000 Hz) using acoustic stimuli.

Note that although cochlear implant 100 has been described as comprising 22 electrodes 142, some of these electrodes might produce non auditory percepts (e.g. facial stimulation or pain) and so would not be included in map data 222. Electrodes used in a map are referred to as “selected” or “activated” channels. It should also be appreciated that although there is a one-to-one correspondence between electrode 142 and channels, in alternative embodiments there is no such correspondence. Accordingly, map data 222 may also include data allocating each frequency band to, for example, an electrode pair.

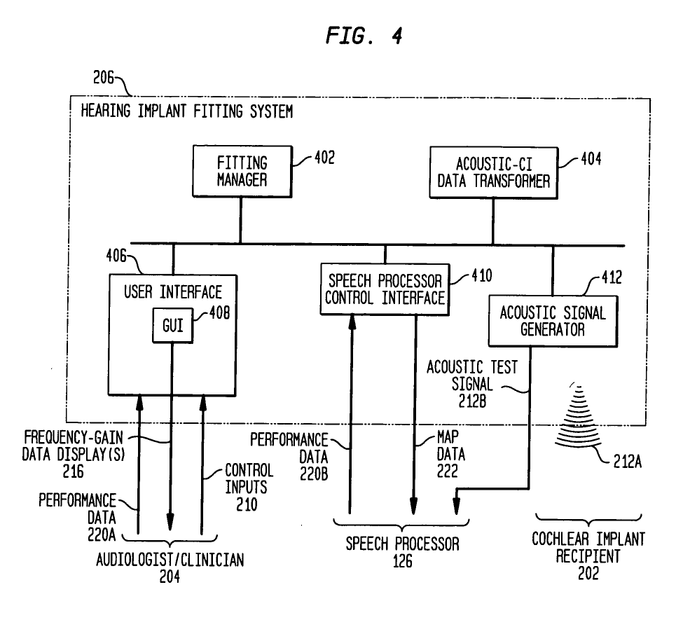

FIG. 4 is a high-level functional block diagram of hearing implant fitting system 206 according to one embodiment of the present invention. The primary components and operative aspects of fitting system 206 are shown in block diagram form for ease of description, and are described herein. The primary components are interoperably coupled to perform fitting operations. In the exemplary embodiment shown in FIG. 4, the components are shown as being coupled by a communications bus. However, it is to be understood that the components of fitting system 206 may be connected in any manner suitable for the particular application.

Fitting manager 402 performs fitting operations and controls the other components shown in FIG. 4. The operation and other aspects of fitting manager 402 are known in the art, and are not further described herein.

Acoustic signal generator 412 generates acoustic test signal 212 as noted above. Acoustic test signal 212 is generated by fitting system 206 as part of the fitting process 300 implemented to configure or fit cochlear implant 100 to recipient 202. Acoustic test signal 212 can be sent by free field transmission, and is depicted in FIG. 4 as acoustic test signal 212A. Free field transmission refers to sending acoustic signals through air to cochlear implant recipient 202. Alternatively, acoustic test signal 212 can be sent to recipient 202 via a channeling, directing or other intermediate device. In FIG. 4, for example, acoustic test signal 212B is provide electronically to cochlear implant recipient 202 via speech processor 126. Acoustic test signals 212A and 212B are generally and collectively referred to herein as acoustic test signals 212. It is to be understood that acoustic test signal 212 can be transmitted by acoustic signal generator 312 through the aforementioned techniques, as well as any techniques or devices now or later developed.

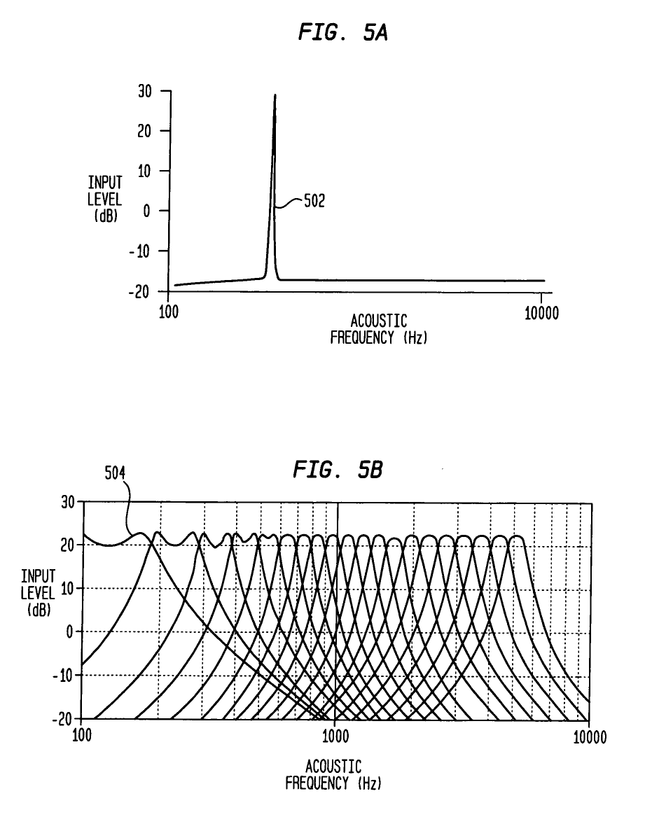

Acoustic test signal 212 can be either a pure tone, an example of which is depicted in FIG. 5A. As shown therein, a pure tone is one in which the acoustic test signal 212 is composed primarily of a single frequency or very narrow frequency range. Alternatively, acoustic test signal 212 may be a composition test signal comprising a narrow or broad range of frequencies. A composition frequency test signal is one in which the acoustic test signal 212 is composed of multiple frequency ranges or a selection of one or more of the multiple frequency ranges, as depicted in FIG. 5B. This latter embodiment may be desirable since during normal operation of cochlear implant 100, the incoming sound signal typically contains a wide range of frequencies.

As noted, fitting system 206 is operably coupled to cochlear implant 100 via data communication link 208. In the embodiment shown in FIG. 2 fitting system 206 includes a speech processor control interface 410 to provide such interoperability. Performance data 220B can be sent from cochlear implant 100 to fitting system 206. Also, data such as the mapping data 222 can be sent to cochlear implant 100 through speech processor interface 410.

User interface 406 can include any interface which is used by audiologist/clinician 204 to communicate with fitting implant system 206. The audiologist/clinician 204 can provide input using any one or combination of known methods, including a computer keyboard, mouse, voice-responsive software, touch-screen, retinal control, joystick, and any other data entry or data presentation formats now or later developed.

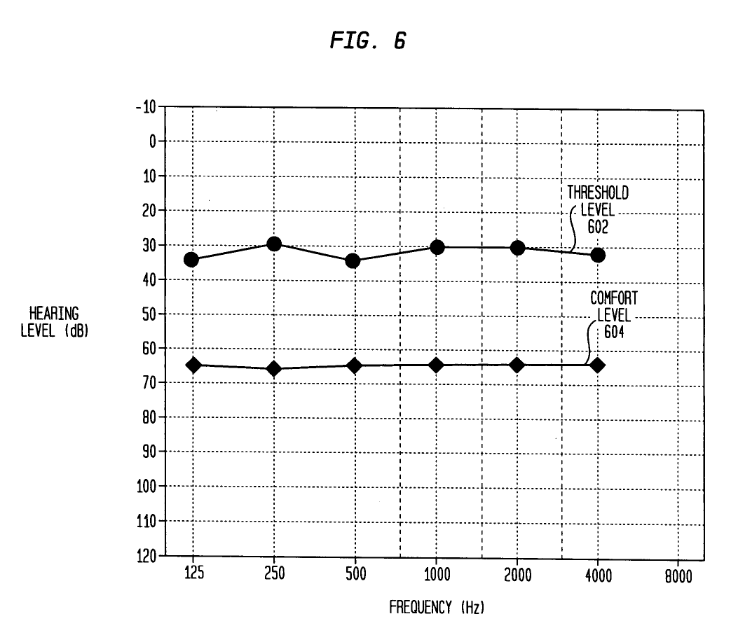

In the embodiment illustrated in FIG. 4, user interface 406 includes a graphical user interface (GUI) 408 which is displayed on display device 214, as noted above. As noted, user interface 406 provides and receives acoustic-based data; that is, data which is in the form commonly used by audiologists to fit hearing aids. In this exemplary embodiment, such data is presented as frequency-gain data displays 216. According to one embodiment, a frequency-gain data display 216 as depicted in FIG. 6 is displayed on display device 214. As shown, audiologist/clinician 204 conducts the fitting process by providing acoustic-based control inputs 210 for which the cochlear implant recipient 202 and/or cochlear implant 100 provides performance data 220. Through this fitting process threshold levels 602 and comfort levels 604 are set as displayed in GUI 408. Although threshold levels 602 and comfort levels 604 are represented as line graphs in FIG. 6, it should appreciated that such frequency-gain data may be represented in other forms including, but not limited to, bar graphs, tables, histograms, drop-down menus, radio buttons, text boxes, or other method for displaying such data.

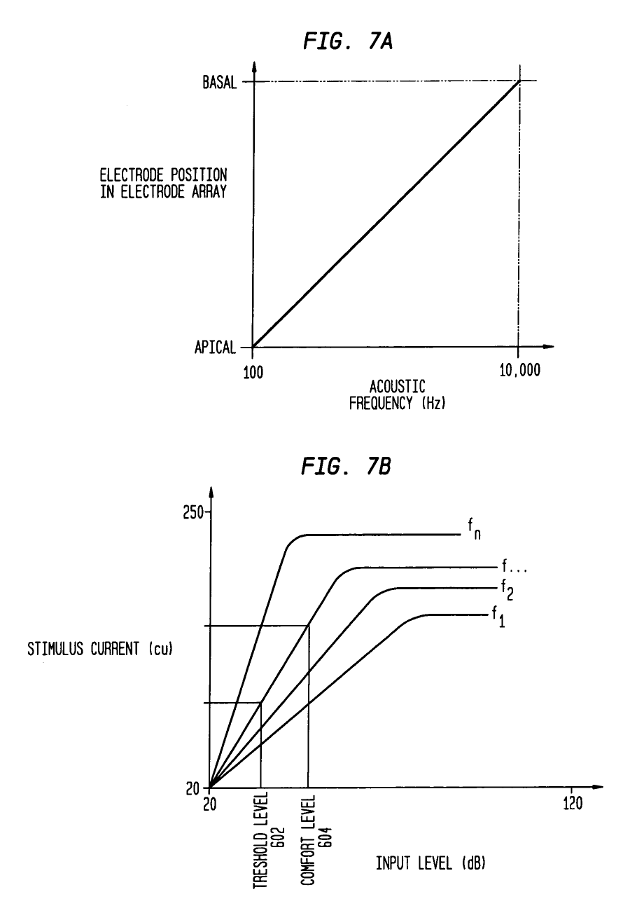

As noted, cochlear implant 100 is optimized for each recipient 202 by conducting one or more fitting sessions in which the recipient's map data 202 is generated. According to the teachings of the present invention, during fitting process 300, an audiologist or clinician provides acoustic-based control inputs 210 in user interface 406 provides acoustic-based data display such as the noted frequency-gain displays noted above. Through fitting process 300, threshold levels 602 and comforts levels 604 are determined for each frequency band or electrode 142 in acoustic terms of level (dB) versus frequency (Hz). As noted, fitting system 206 transforms such acoustic-based data to implant-based map data 222 for transmission to speech processor 126. For example, in the above description, threshold and comfort levels were set by audiologist/clinician 204 by adjusting the gain for each frequency band while the resulting map data 222 is provided to speech processor 126 in terms of gain versus current units (cu) for each frequency band. This is described in further detail below with reference to FIGS. 7A and 7B.

In the exemplary embodiment illustrated in FIG. 4, fitting system 206 comprises an acoustic-cochlear implant (CI) data transformer 404 that transforms the acoustic-based map data to implant-based map data 222. To perform such transformations, data transformer 404 determines which electrode 142 the particular frequency has been allocated, and the current unit (cu) corresponding to the gain selected or set for that frequency by audiologist/clinician 204. Data transformer 404 stores and/or accesses information establishing the relationship between these values. Such information may be stored in any manner suitable for the particular application, including, programmed in an ASIC or other computer hardware, software code, data tables stored in memory, etc.

The information establishing the relationship between these values is represented in two line graphs illustrated in FIGS. 7A and 7B. FIG. 7A is a line graph illustrating the relationship between frequency (Hz) and electrode position on electrode array 144 when implanted in cochlea 116. For each frequency of acoustic test signal 212 selected by the audiologist/clinician 204, data transformer 404 uses the relationship depicted in FIG. 7A to select the electrode 142 that will be positioned in the region of cochlear 116 corresponding to that frequency.

FIG. 7B is a line graph illustrating the amplitude (cu) of the stimulation signal required to cause a particular loudness percept (dB) for each frequency band f₁ . . . fₙ. In this example, the horizontal axis represents amplitudes (in dB) of acoustic test signals 212, and the vertical axis represents logarithmic stimulus current levels from about 2 microamps to 2 milliamps. The pulse width in this example is fixed at 9.6 micro seconds. Using the relationships shown in FIG. 7B, data transformer 404 determines the current amplitude of the stimulation signal corresponding to the threshold and comfort levels 602, 604 selected by the audiologist/clinician 204 for each frequency band. Alternatively, the same function may be implemented to represent the relationship between stimulation signal amplitude and loudness percept regardless of frequency.

Based on the above, map data 222 is generated by data transformer 404 for application to speech processor 126. Map data 222 includes, for example, the threshold and comfort stimulation current levels for each electrode 142 corresponding to a selected frequency band. As noted, since this version of map data 222 is in a form suitable for the implemented cochlear implant 100, the map data is referred to as implant-based map data.

Further features of the present invention are described in U.S. Provisional Application No. 60/650,148, entitled “Hearing Implant Programming Technique,” filed Feb. 7, 2005, which is hereby incorporated by reference herein in its entirety. Other information may be found in (1) Gitte Keidser, et al. “Using the NAL-NL1 prescriptive procedure with advanced hearing aids.” National Acoustic Laboratories. Mar. 2, 2006, pages 1-10. (Reprinted with permission from The Hearing Review, November 1999.); and (2) Teresa Y. C. Ching, et al. “RECD, REAG, NAL-NL1: Accurate and practical methods for fitting non-linear hearing aids to infants and children.” National Acoustic Laboratories, Reprinted with permission from The Hearing Review, August 2002, vol. 9, no. 8, pages 12-20, 52.

All documents, patents, journal articles and other materials cited in the present application are hereby incorporated by reference herein.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart therefrom.

1. A fitting system for fitting a prosthetic hearing implant for a recipient, the implant having an array of electrodes implanted in the recipient's a cochlear, comprising:
a user interface configured to provide acoustic-based fitting data and receive acoustic-based control inputs;
an acoustic test signal generator configured to provide the recipient acoustic test signals generated in response to said acoustic-based control inputs; and
a data transformer configured to transform said acoustic-based fitting data to implant-based fitting data for use by the hearing implant.

2. The system of claim 1, wherein said acoustic test signals comprise at least one of either a pure tone acoustic test signal comprising a narrow band of one or more frequencies, and a composition acoustic test signal comprising a broad range of a plurality of frequencies.

3. The system of claim 1, wherein said acoustic-based fitting data comprises:
frequency-gain fitting data.

4. The system of claim 3, wherein said frequency-gain fitting data comprises:
threshold and comfort levels for each selected frequency of each said acoustic test signal, wherein said threshold and comfort levels are in terms of decibels versus frequency.

5. The system of claim 3, wherein said frequency-gain fitting data comprises:
threshold and comfort levels for each selected frequency of each said acoustic test signal, wherein said threshold and comfort levels are in terms of current units versus decibels for each of said selected frequencies.

6. The system of claim 1, wherein said acoustic test signals are transmitted via at least one of either free field or an intermediate device.

7. A method for fitting a prosthetic hearing implant for a recipient, said implant having an array of electrodes implanted in the recipient's cochlear, the method comprising:
establishing a data communication link between said prosthetic hearing implant and a fitting system;
generating acoustic-based map data for said prosthetic hearing implant;
transforming said acoustic-based map data into implant-based map data; and
applying said implant-based map data to said prosthetic hearing implant.

8. The method of claim 7, wherein said establishing a data communication link comprises:
calibrating one or more of said electrodes on said array of electrodes with said fitting system.

9. The method of claim 7, wherein data communication link comprises a bi-directional data communication link.

10. The method of claim 7 wherein applying implant-based map data comprises:
sending said implant-based map data to said prosthetic hearing implant via said data communication link.

11. The method of claim 10, wherein applying implant-based map data furtherb comprises:
applying said implant-based map data to said prosthetic hearing implant.

12. The method of claim 7, wherein said generating of said acoustic-based map data comprises:
generating at least one acoustic test signal to said prosthetic hearing implant;
displaying acoustic-based displays; and
receiving acoustic-based operator inputs.

13. The method of claim 12, wherein said at least one acoustic test signal is generated by an acoustic signal generator.

14. The method of claim 12, wherein said generating of said acoustic-based map data comprises:
setting a threshold level and a comfort level for the recipient via said acoustic-based user interface.

15. The method of claim 12, wherein said receiving acoustic-based operator inputs comprises:
receiving threshold and comfort levels.

16. The method of claim 7, wherein said generating of acoustic-based map data comprises:
storing said acoustic-based map data for threshold and comfort levels for each of one or more frequencies.

17. The method of claim 7, wherein said transforming of acoustic-based map data comprises:
determining which of said array of electrodes corresponds to at least one frequency band comprising one or more frequencies; and
determining the current level corresponding to the threshold and comfort levels for each of at least one frequency band.

18. The method of claim 17, wherein said transforming of said acoustic-based map data comprises:
storing said map data in a memory device.

19. The method of claim 17, wherein said transforming of said acoustic-based map data comprises:
storing said map data in a memory device on said fitting system.

20. The method of claim 17, wherein said transforming of said acoustic-based map data comprises:
storing said map data in a memory device on said prosthetic hearing implant.

21. A fitting system for fitting a prosthetic hearing implant for a recipient, said implant having an array of electrodes implanted in said recipient's a cochlear, comprising:
providing means for providing acoustic-based map data and receiving acoustic-based control inputs;
generating means for generating acoustic test signals in response to acoustic-based control inputs;
transforming means for transforming said acoustic-based map data to implant-based map data for application to said prosthetic hearing implant.

22. The system of claim 21 wherein said means for generating acoustic-based test signals comprises:
generating means which sends test signals by free field or via intermediate device to said prosthetic hearing implant and said fitting system.

23. The system of claim 21 wherein said means for generating acoustic test signals sends pure tone or composition frequency test signal.

24. The system of claim 21 wherein said means for providing acoustic-based fitting data displays frequency-gain data.
